(19) United States
(12) Patent Application Publication
(10) Pub. No.: US 2002/0192696 A1
(43) Pub. Date: Dec. 19, 2002

(54) Using Cdc6 as therapeutic, prognostic and diagnostic targets for cell growth/proliferation

(76) Inventor: Ambrose Y. Jong, Arcadia, CA (US)

Correspondence Address: Raymond Y. CHAN, Suite 128, 108 N. Ynez Ave., Monterey Park, CA, 91754, US

(21) Appl. No.: 10/170,488

(22) Filed: Jun. 12, 2002

Related U.S. Application Data
(60) Provisional application No. 60/298,585, filed on Jun. 14, 2001.

(51) Int. Cl. C12Q 1/68

(52) U.S. Cl. 435/6

(57) ABSTRACT

Using Cdc6 as therapeutic, prognostic and diagnostic targets for cell growth/proliferation. The unique features of Cdc6 allow anti-Cdc6 treatments to have affect on cancer cells, not on normal cells. Antisense anti-Cdc6 DNA technology is used to test Cdc6 as a target for growth inhibition and shows that the abrogation of Cdc6 mRNA in rapid cell results in death of prostate PC-3, DU145 and LNCaP cancer cells. The study demonstrated the abrogation of Cdc6 mRNA functions in prostate cancer cells could be an extremely effective way to block the cancer cell proliferation. Anti-Cdc6 treatment can be used alone, in combination, or in synergy with currently used therapeutic drugs or radiation therapies. Thus, the Cdc6 gene, mRNA and protein can also be a useful marker for diagnosis, prognosis, or therapy of cell proliferation.

### CROSS REFERENCE OF RELATED APPLICATION

This application is a regular application of a provisional application, application No. 60/298,585, filed Jun. 14, 2001

### BACKGROUND OF THE PRESENT INVENTION

1. Field of Invention

This invention relates to targeted gene therapy. In particular, this invention relates to the use of human Cdc6 gene, mRNA, or protein as novel target sites for cancer therapy, diagnosis, and prognosis.

2. Description of Related Arts

Cancer is a major killer among diseases in the United States and all over the world. Statistically, one out of four people will develop some kinds of cancer in his/her lifetime. Despite the current standard treatments (surgery, radiation, and chemotherapy), there is currently no effective ways for most cancer treatments and many patients eventually succumb to the disease. Thus, towards development of new targets and novel strategies, at both early and advanced stages, is a continuous effort for the cancer treatment.

Cancer is a highly proliferative cell. The regulatory mechanism of cell growth in cancer cells, at least in part, is out of control. Many cell proliferative genes encode cell cycle kinases and their regulators, growth factors, receptors, or proteins of signal transduction pathways. These pathways are inherently redundant, so that most of these genes may not be a reliable marker or therapeutic target for neoplastic cells. However, cell cycle and signaling pathways converge at the point of initiation of DNA replication. Thus, the control of cell proliferation at the initiation of DNA replication is a highly specific and effective way for cancer treatment. Indeed, most current cancer chemotherapies are targeting at DNA synthesis. While the blockage DNA synthesis is useful, the control of upstream targets at the initiation step has little been explored.

Recent recombinant DNA techniques and other advanced biotechnological approaches make it possible in search of novel targets at the initiation step of DNA replication for cancer therapy. Key components such as ORC (Origin recognition complex), Cdc6, MCM protein (Minichromosome maintenance proteins) are essential for the regulation of initiation of DNA replication in all eukaryotes including humans. ORC, a six tightly associated protein complex (Orc1 to Orc6) was first identified in the budding yeast. It binds to replication origins throughout the cell cycle and serves as a loading pad for other initiation proteins. Current models view the initiation of DNA replication in eurkaryotic cells as a two-step process: a competence step and an activation step (FIG. 1). In the G2 phase of the cell cycle, ORC is bound to the DNA replication origin in post-replicative complex (post-RC or competent) state. Since only the post-RC can be observed in arrested Cdc6 mutant in yeast, it has been suggested that Cdc6 protein is required for establishment and maintenance of pre-RC. Late in G1 phase, the MCM complex is loaded, and then Cdc6 is removed, leading to an activated state. This step is controlled by cell cycle kinases. Cdc6 is subsequently degraded in the regulated proteolytic pathway. After the loading of Cdc45 and redistribution of the MCM complex, initiation of DNA replication can occur. The action of Cdc7-Dbf4 is essential at this step.

Cdc6 is an attractive target because human cells have only one member of the Cdc6 gene. There are no other functional redundant genes or isoforms in human cells. Thus, Cdc6 could be a highly specific target for chemo-and/or gene-therapies. The Cdc6 gene encodes a low abundant, short half-life protein, and the effect of down-regulation of Cdc6 could therefore be potent, if any treatment applies. In some types of tumors, Cdc6 mRNA is not detectable in normal cells, but markedly expressed in the proliferative cancer cells.

This unique feature implies that anti-Cdc6 treatments may have an affect on cancer cells, not on normal cells. Taking advantage of these properties, antisense anti-Cdc6 DNA technology is used to test Cdc6 as a novel target for growth inhibition. These studies indicated that abrogation of Cdc6 mRNA resulted in rapid cell death of prostate PC-3, DU145 and LNCaP cancer cells. The study demonstrated the abrogation of Cdc6 mRNA functions in prostate cancer cells could be an extremely effective way to block the cancer cell proliferation. This application is not limited to tested prostate cancer cell; any other highly localized tumors (brain, bladder, neuroblastoma, etc.) can be treated in a similar way. The anti-Cdc6 mRNA treatment can be extended to anti-Cdc6 protein functions, such as the use of interfering peptides to block the Cdc6 protein functions. To generate mutant Cdc6 gene that has a dominant negative effect on cell growth is another way for anti-Cdc6 gene therapy. Since Cdc6 is a low copy, short half-life protein, and the Cdc6 mRNA and protein level will decrease rapidly after any anti-Cdc6 treatment. This feature allows us to use Cdc6 as the diagnostic and prognostic marker of cell proliferation.

In summary, the initiation of DNA replication is a very complicated process, and the Cdc6 is the major trigger of this process. It is speculated that any perturbation of Cdc6 gene function may result in deteriorate effect on cell growth and even lead to the programmed cell death (apoptosis). The effect may be more prominent in the highly proliferative cells, including cancer cells. Several anti-Cdc6 mRNA studies which can rapidly induce cell program death (apoptosis) in cancer cells (FIG. 3B) have been performed.

The following listed publication illustrates the background art of the present invention:

Aparicio O M, Weinstein D M, Bell S P. 1997. Components and dynamics of DNA replication complexes in S. cerevisiae: redistribution of MCM proteins and Cdc45 p during S phase. Cell 91:59-69

Bell S P, Kobayashi R, Stillman B. 1993. Yeast origin recognition complex functions in transcription silencing and DNA replication. Science 262:1844-9

Berger C, Strub A, Staib C, Lepke M, Zisimopoulou P, Hoehn K, Nanda I, Schmid M, Grummt F. 1999. Identification and characterization of a mouse homolog to yeast Cdc6 p. Cytogenet. Cell Genet. 86:307-16

Bogan J A, Natale D A, Depamphilis M L. 2000. Initiation of eukaryotic DNA replication: conservative or liberal? J Cell Physiol 184:139-50

Castelli J, Wood K A, Youle R J. 1998. The 2-5A system in viral infection and apoptosis. Biomed. Pharmacother. 52:386-90

Cocker J H, Piatti S, Santocanale C, Nasmyth K, Diffley J F. 1996. An essential role for the Cdc6 protein in forming the pre-replicative complexes of budding yeast. Nature 379:180-2

Coleman T R, Carpenter P B, Dunphy W G. 1996. The Xenopus Cdc6 protein is essential for the initiation of a single round of DNA replication in cell-free extracts. Cell 87:53-63

Cotter F E, Waters J, Cunningham D. 1999. Human Bc1-2 antisense therapy for lymphomas. Biochim. Biophys. Acta 1489:97-106

Detweiler C S, Li J J. 1997. Cdc6 p establishes and maintains a state of replication competence during G1 phase. J Cell Sci. 110 (Pt 6):753-63

Donovan S, Harwood J, Drury L S, Diffley J F. 1997. Cdc6 p-dependent loading of Mcm proteins onto pre-replicative chromatin in budding yeast. Proc Natl. Acad. Sci. U.S.A. 94:5611-6

Drury L S, Perkins G, Diffley J F. 1997. The Cdc4/34/53 pathway targets Cdc6 p for proteolysis in budding yeast. EMBO J 16:5966-76

Drury L S, Perkins G, Diffley J F. 2000. The cyclin-dependent kinase Cdc28p regulates distinct modes of Cdc6 p proteolysis during the budding yeast cell cycle. Curr. Biol. 10:231-40

Dutta A, Bell S P. 1997. Initiation of DNA replication in eukaryotic cells. Annu. Rev. Cell Dev. Biol. 13:293-332

Ferrari S, Baserga R. 1987. Oncogenes and cell cycle genes. Bioessays 7:9-13 Fujita M, Yamada C, Goto H, Yokoyama N, Kuzushima K, Inagaki M, Tsurumi T. 1999. Cell cycle regulation of human CDC6 protein. Intracellular localization, interaction with the human mcm complex, and CDC2 kinase-mediated hyperphosphorylation. J Biol. Chem. 274:25927-32

Hartwell L H. 1976. Sequential function of gene products relative to DNA synthesis in the yeast cell cycle. J Mol. Biol. 104:803-17

Hateboer G, Wobst A, Petersen B O, Le Cam L, Vigo E, Sardet C, Helin K. 1998. Cell cycle-regulated expression of mammalian CDC6 is dependent on E2F. Mol. Cell Biol. 18:6679-97

Jiang W, Wells N J, Hunter T. 1999. Multistep regulation of DNA replication by Cdk phosphorylation of HsCdc6. Proc Natl. Acad. Sci. U.S.A 96:6193-8

Jong A, Young M, Chen G C, Zhang S Q, Chan C. 1996. Intracellular location of the Saccharomyces cerevisiae CDC6 gene product. DNA Cell Biol. 15:883-95

Jong A Y, Wang B, Zhang S Q. 1995. Pulsed field gel electrophoresis labeling method to study the pattern of Saccharomyces cerevisiae chromosomal DNA synthesis during the G1/S phase of the cell cycle. Anal. Biochem. 227:32-9

Kelly T J, Brown G W. 2000. Regulation of chromosome replication. Annu. Rev. Biochem. 69:829-80

Khuri F R, Kurie J M. 2000. Antisense approaches enter the clinic. Clin. Cancer Res 6:1607-10

Kondo S, Kondo Y, Li G, Silverman R H, Cowell J K. 1998. Targeted therapy of human malignant glioma in a mouse model by 2-5A antisense directed against telomerase RNA. Oncogene 16:3323-30

Lee D G, Bell S P. 1997. Architecture of the yeast origin recognition complex bound to origins of DNA replication. Mol. Cell Biol. 17:7159-68

Lopes de Menezes D E, Hudon N, McIntosh N, Mayer L D. 2000. Molecular and pharmacokinetic properties associated with the therapeutics of bcl 2 antisense oligonucleotide G3139 combined with free and liposomal doxorubicin. Clin. Cancer Res 6:2891-902

Muzi-Falconi M, Kelly T J. 1995. Orpl, a member of the Cdc18/Cdc6 family of S-phase regulators, is homologous to a component of the origin recognition complex. Proc Natl. Acad. Sci. U.S.A 92:12475-9

Neidle S, Kelland L R. 1999. Telomerase as an anti-cancer target: current status and future prospects. Anticancer Drug Des 14:341-7

Nishitani H, Nurse P. 1995. p65cdc18 plays a major role controlling the initiation of DNA replication in fission yeast. Cell 83:397-405

Orr R M, O'Neill C F. 2000. Patent review: therapeutic applications for antisense oligonucleotides 1999-2000. Curr. Opin. Mol. Ther. 2:325-31

Perkins G, Diffley J F. 1998. Nucleotide-dependent prereplicative complex assembly by Cdc6 p, a homolog of eukaryotic and prokaryotic clamp-loaders. Mol. Cell 2:23-32

Petersen B O, Lukas J, Sorensen C S, Bartek J, Helin K. 1999. Phosphorylation of mammalian CDC6 by cyclin A/CDK2 regulates its subcellular localization. EMBO J 18:396-410

Piatti S, Lengauer C, Nasmyth K. 1995. Cdc6 is an unstable protein whose de novo synthesis in G1 is important for the onset of S phase and for preventing a ‘reductional’ anaphase in the budding yeast Saccharomyces cerevisiae. EMBO J 14:378899-99

Seki T, Diffley J F. 2000. Stepwise assembly of initiation proteins at budding yeast replication origins in vitro. Proc Natl. Acad. Sci. U.S.A 97:14115-20

Steiner M S, Gingrich J R. 2000. Gene therapy for prostate cancer: where are we now? J Urol. 164:1121-36

Takei Y, Yamamoto K, Tsujimoto G. 1999. Identification of the sequence responsible for the nuclear localization of human Cdc6. FEBS Lett. 447:292-6

Williams R S, Shohet R V, Stillman B. 1997. A human protein related to yeast Cdc6 p. Proc Natl. Acad. Sci. U.S.A 94:142-7

Yan Z, DeGregori J, Shohet R, Leone G, Stillman B, Nevins J R, Williams R S. 1998. Cdc6 is regulated by E2F and is essential for DNA replication in mammalian cells. Proc Natl. Acad. Sci. U.S.A 95:3603-8

Ying S Y, Lin S. 1999. High-performance subtractive hybridization of cDNAs by covalent bonding between specific complementary nucleotides. Biotechniques 26:966-2,979

Ying S Y, Chuong C M, Lin S. 1999. Suppression of activin-induced apoptosis by novel antisense strategy in human prostate cancer cells. Biochem. Biophys. Res Commun. 265:669-73

Yuen A R, Sikic B I. 2000. Clinical studies of antisense therapy in cancer. Front Biosci. 5:D588-D593

Zhou C, Huang S H, Jong A Y. 1989. Molecular cloning of Saccharomyces cerevisiae CDC6 gene. Isolation, identification, and sequence analysis. J Biol. Chem. 264:9022-9

Zhou C, Jong A. 1990. CDC6 mRNA fluctuates periodically in the yeast cell cycle. J Biol. Chem. 265:19904-9

Zhou C, Jong A Y. 1993. Mutation analysis of Saccharomyces cerevisiae CDC6 promoter: defining its UAS domain and cell cycle regulating element. DNA Cell Biol. 12:363-70

Zwerschke W, Rottjakob H W, Kuntzel H. 1994. The Saccharomyces cerevisiae CDC6 gene is transcribed at late mitosis and encodes a ATP/GTPase controlling S phase initiation. J Biol. Chem. 269:23351-6

### SUMMARY OF THE PRESENT INVENTION

An object of the present invention is to use anti-sense oligonucleotides (ASO) or any modified ASO (modified nucleotides, 2.5-Ade, others) against Cdc6 mRNA in cancer cells.

Another object of the present invention is to use RNA interfering (RNAi), DRNA (DNA and RNA hybrid) techniques against Cdc6 mRNA in cancer cells.

Another object of the present invention is to use anti-Cdc6 peptides (interfering peptides) to block Cdc6 gene functions in cancer cells.

Another object of the present invention is to use dominant-negative mutant Cdc6 genes for gene therapy in cancer cells.

Another object of the present invention is to detect Cdc6 mRNA using Northern blotting, RT-PCR, microarray, etc., in cancer cells for diagnostic or prognostic purposes.

Another object of the present invention is to detect Cdc6 protein using Western blotting, histological staining, imaging analysis, proteomic analysis, microarray, etc., in cancer cells for diagnosis or prognosis purposes.

Another object of the present invention is to use Cdc6 as the therapeutic target for cancer patients, including using anti-sense oligonucleotides (ASO), or any modified ASO, RNA interfering (RNAi) methods, interfering peptides, DRNA (DNA and RNA hybrid) against Cdc6 functions, etc.

Another object of the present invention is to treat cancers, including but not limited to, prostate cancer, brain tumors, lung cancer, cervix cancer, ovary cancer, kidney cancer, bladder cancer, neuroblastomas and any other highly proliferative cells.

Another object of the present invention is to use Cdc6 protein or mRNA as the diagnostic target for cancer cells, or cell growth index in various cancers.

Another object of the present invention is to use Cdc6 protein or mRNA as the prognostic target for cancer cells, or cell growth index.

### BRIEF DESCRIPTION OF THE DRAWINGS

FIG. 1 is a diagram summarizing Human Cdc6 gene function at the initiation of DNA synthesis.

FIG. 2A is a diagram summarizing the expression levels of Human Cdc6 mRNA in different tissues.

FIG. 2B is a diagram illustrating the expression of Cdc6 in matched normal and tumor samples.

FIG. 3A is a diagram illustrating anti-sense Cdc6 oligo chimeric with RNase L Activator: (2-5) Ade.

FIG. 3B is a diagram illustrating Trypan blue positive DU 145 cells after anti-Cdc6 treatment.

FIG. 4A is a diagram illustrating TUNEL assay is used for showing apoptotic cell after anti-Cdc6 treatment.

FIG. 4B is a diagram illustrating Protein blot is used for showing the disappearance of Cdc6 protein after anti-Cdc6 treatment.

### DETAILED DESCRIPTION OF PREFERRED EMBODIMENT

As shown in FIG. 1, the formation of pre-replicative complex at replication origins starts from the loading of Cdc6 at replication origins, leading into the competent state. The removal of Cdc6 from ORC-origin prevents DNA re-replication and leads to the activation of DNA synthesis. Thus, Cdc6 is the major trigger of the initiation of DNA synthesis.

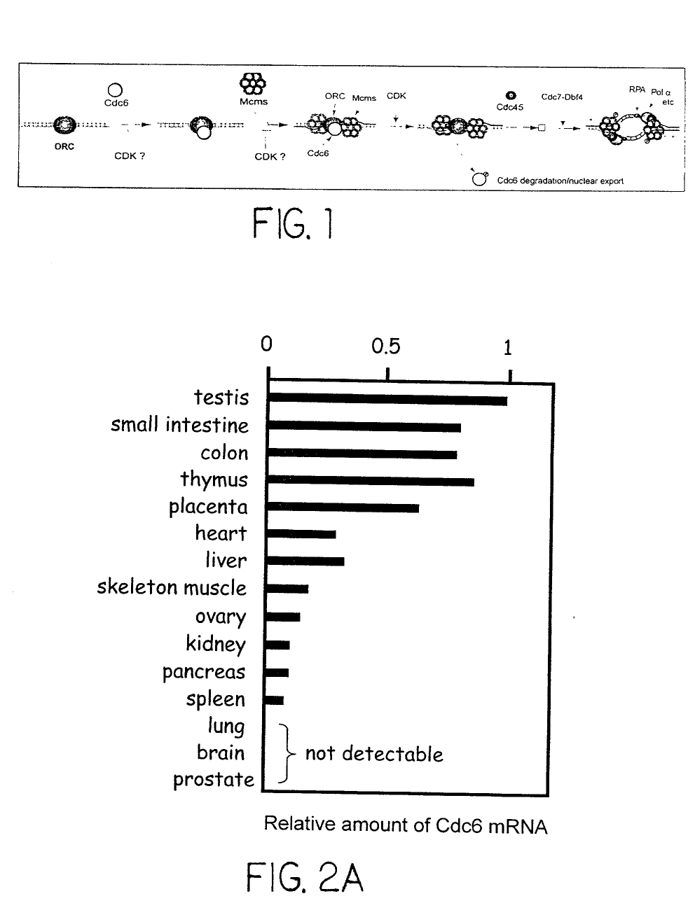

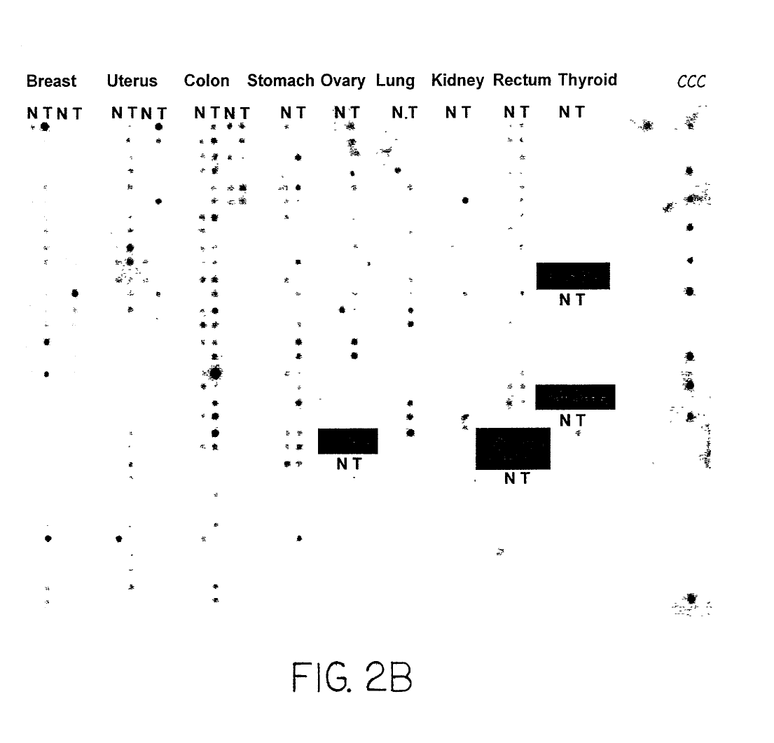

As shown in FIG. 2A and 2B, the tissue blot and the cancer blot, purchased from Clontech, were probed with human Cdc6 riboprobe. The relative intensity of Cdc6 spots from different tissues was scanned by PhosphoImager™ (Bio-Rad), and normalized to testis reading as 1.0 in FIG. 2A. In 2B, cancer types are listed on the top of the matched normal (N) and tumor (T) pairs. CCC stands for “control cancer cells”, the positive controls on the blot.

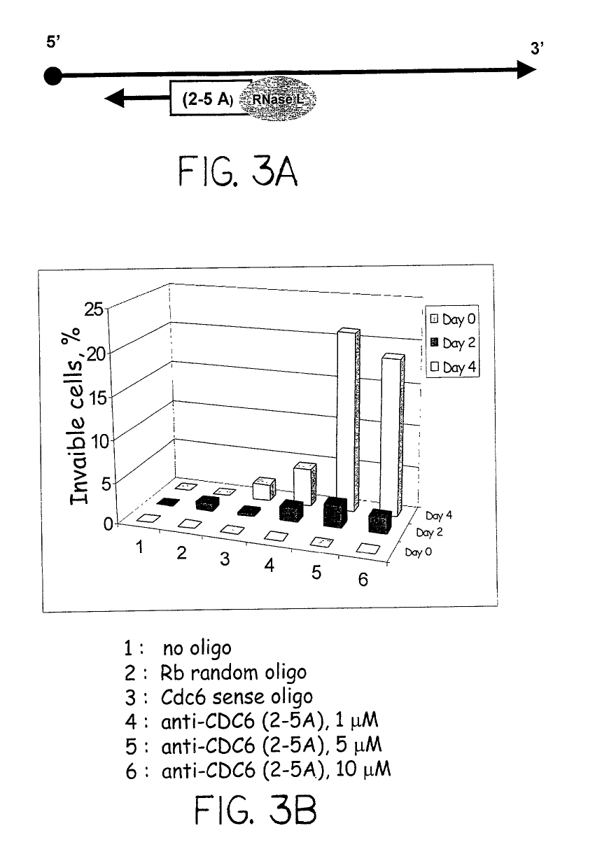

As shown in FIG. 3A, the anti-Cdc6 ASO that contains 18 nucleotides is complementary to the initiation codon of Cdc6 (marked as *) and links to modified oligo 2′,5′-olgioadenylate (2,5A). The (2,5A) moiety can recruit and activate endogenous RNase L, resulting in digestion of Cdc6 and mRNA.

As shown in FIG. 3B, prostate cancer cells DU145 were treated with different oligonucleotides. The dead DU145 cells were counted by trypan blue staining under microscope at day 0, 2, and 4. Lane 1, no oligo; lane 2, RB random oligo; lane 3, Cdc6 sense oligo; lanes 4-6: 1 μM, 5 μM, and 10 μM anti-Cdc6 ASO-(2-5A) oligo, individually.

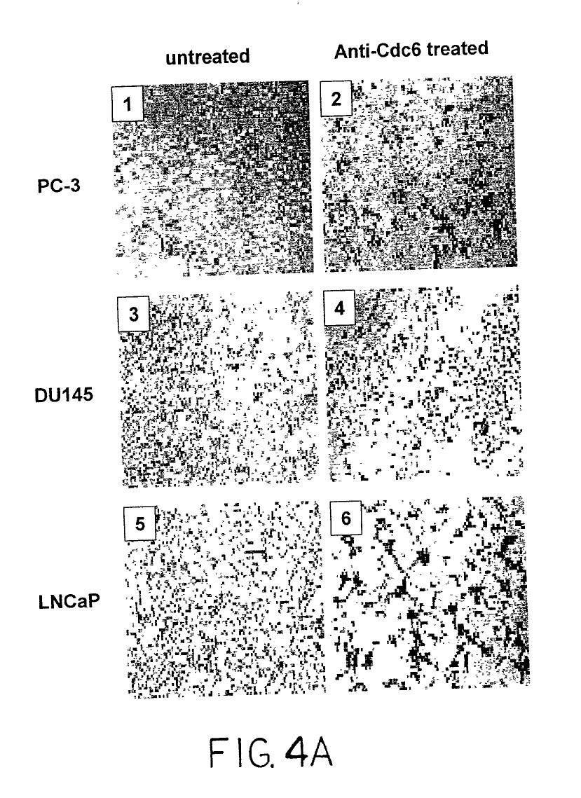

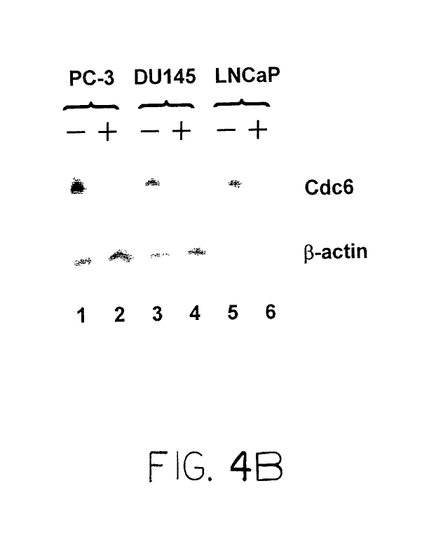

As shown in FIG. 4A, prostate cancer cell lines PC-3, DU145, LNCaP are used. Although different prostate cancer cells display different morphology, they all showed significant DNA fragmentation after anti-Cdc6 treatment, as evidenced by dark brown dots in the anti-Cdc6 treated cells.

As shown in FIG. 4B, the same set of samples was subjected to the Western blotting analysis using anti-Cdc6 monoclonal antibody (Sigma, DCS-180). The detected Cdc6 proteins were shown on the upper panel and β-actin was used as the control. “+”: treated cells; “−”: untreated cells.

Cdc6 from yeast to human have been studied for more than 15 years. The accumulated information suggests that human Cdc6 can be a novel target for cancer therapy, diagnosis and prognosis. Some unique features of human Cdc6 provide many advantages over current methodologies. Below are summaries of these features.

Human genome contains one Cdc6 gene. Genomic sequence analysis does not reveal other Cdc6 isoforms. Human Cdc6 is not a member of any gene family. The single-copy makes Cdc6 a highly specific target for chemo-or gene-therapy. The most structurally related gene is the Orc1, another origin replication protein. However, the conserved regions are only observed in the ATP-binding domains. In yeast, deprivation of Cdc6 gene functions results in lethal phenotype. The results suggest that yeast Cdc6 is a crucial regulatory protein playing a vital role in the G1-to-S transition. It is believed the human Cdc6 also plays essential roles in proliferative cells. Thus, the single-copy and its essential function may make Cdc6 a highly effective target for cancer therapy. Cdc6 is a relatively short-lived protein. In budding yeast, the half-life of Cdc6 is approximately 5-min. Human Cdc6 protein has a similar property with an estimated half-life of 2 hrs in both transformed and primary asynchronized cells lines. In synchronized HeLa cells, the half-life of Cdc6 in early G1 is estimated to be 15˜30 min. Cdc6 is a low abundant protein. It will be degraded by APC-dependent proteolysis system before S phase. Human Cdc6 was absent in cells released for the G1/S block for 12-hr. Thus, in each cell cycle Cdc6 will be newly synthesized to exert its gene function at replication origin during G1 phase, and then the majority of Cdc6, if not all, will be degraded or removed from its functional site before S phase (FIG. 1). The single isoform, low abundance, short half-life and narrow functional window in the cell cycle suggest that Cdc6 could be an extremely sensitive target for cancer therapy.

Like any other gene, Cdc6 expresses different levels in different tissues. Interestingly, Cdc6 does not express in normal prostate, lung and brain tissues. In contrast, it markedly expresses in proliferative cancer cells (please see FIG. 2A). This differential expression provides an extraordinary chance to manipulate its gene level for the control of cell proliferation. Furthermore, the Cdc6 expression in the match normal and tumor pairs from various cancer samples have bee detected. In many tumors, the expression of Cdc6 is much higher in the tumor samples versus to matched normal cells (FIG. 2B). Because of unique properties of the Cdc6 (single-copy, low abundance, essential function, short half-life, and differential expression), the feasibility of targeting Cdc6 as a novel site for prostate cancer therapy is evaluated.

In order to support that human Cdc6 can be a novel target for cell growth inhibition, the detailed Cdc6 functions at ORC-origin must be understood. Some key points of Cdc6 gene functions are summarized as below. Cdc6 was identified as a yeast temperature-sensitive mutant, which caused cell cycle arrested at G1/S phase at 37° C. This is the first time when the full length Cdc6 gene in yeast is cloned by functional complementation and demonstrated that the Cdc6 mutant was defective in DNA synthesis by a molecularly biological approach. Cdc6 has been conserved throughout eukaryotic evolution. The Cdc6 gene has been identified in S. pombe and a variety of metazoans, including mouse, X. laevis, and humans. Genetic studies of S. cerevisiae and S. pombe have established that Cdc6 is required to initiate DNA replication but is not essential for ongoing DNA synthesis. Biochemical studies of Xenous egg extracts have shown that immuno-depletion of Xenopus Cdc6 specifically prevents initiation of chromosomal DNA replication. Similarly, it has been observed that microinjection of human Cdc6 antibody into human cells blocks entry into S phase. It is demonstrated that yeast Cdc6 mRNA expression fluctuated periodically throughout the cell cycle. It is further demonstrated that the 5′-untranslated region of the Cdc6 gene contained an MCB box (Mlul cell cycle box), very similar to those found in the promoter of a group of cell cycle genes involved in DNA replication. It is now known that human Cdc6 gene expression is regulated by transcriptional factor E2F in a similar fashion. Thus, protein synthesis in couple with cell cycle progression may represent the first tier of Cdc6 regulation. It has also been found that nuclear entry of Cdc6 is cell cycle dependent, and mediated by its nuclear localization signal (NLS). In human cells, the subcellular localization of the Cdc6 protein changes during the cell cycle. Human Cdc6 is present in the nucleus during G1 phase but translocates to the cytoplasm at the start of S phase. The translocation of human Cdc6 to the cytoplasm appears to depend on CDK phosphorylation, poses another tier of regulation. The regulation of Cdc6 synthesis and its nuclear translocation provide a threshold concentration at replication origin in the G1 phase to trigger the initiation events (FIG. 1). It is believed that the Cdc6 is the key regulator of DNA replication. It should be an ideal target to control cell proliferation.

In order to demonstrate that human Cdc6 can be a novel target for cancer therapy, (a) it has to be understood that the expression level of Cdc6 gene expression in different tissues, (b) it has to be proven that abrogation of Cdc6 results in drastic effect on cell proliferation. The following issues are addressed:

a) Expression of Human Cdc6 in Different Tissues and Cancer Samples:

One key issue here is that if Cdc6 is an essential component for cell proliferation, one would expect that the expression level of human Cdc6 could be closely related to the cell growth rate. For example, in those tissues that have a higher diving rate, the expression level of human Cdc6 could be higher. Similarly, Cdc6 expression should be elevated in highly proliferative cells such as cancer cells. The expression of human Cdc6 gene in different tissues by Northern blots (FIG. 2A) have been examined. Like most other genes, the Cdc6 mRNA expresses differently in various tissues. Its mRNA can be easily detected in testis, small intestine, colon cells, placenta and thymus. This suggests that the Cdc6 expresses well in these tissues. It expresses moderately in heart and liver cells, skeletal muscle, ovary, kidney, pancreas, and spleen cells. Intriguingly, the Cdc6 mRNA is not detectable in normal prostate, lung and brain cells. Presumably, the highly differentiated tissues such as brain or prostate cells may not actively divide. Thus, the expression of Cdc6 is essentially suppressed in these normal cells.

To further demonstrate the relationship between Cdc6 gene expression and cell proliferation, a Northern blot is performed to detect Cdc6 mRNA in matched normal and tumor samples pairs (FIG. 2B). The differential expression levels between normal and tumor samples in prostate, breast, uterus, ovary, lung, thyroid, cervix, pancreas are significant. Together there is a good correlation between cell proliferation and Cdc6 gene expression level in certain types of cancer cells. Similar results were observed in other proliferative cancer cells such as: rapidly dividing cancer cell lines KB, HeLa, Cac-2, etc (data not shown). These observations suggest that Cdc6 mRNA level is, in general, proportional to the cell proliferation activity.

To use modified antisense oligonucleotide (ASO) against Cdc6 mRNA for growth inhibition studies:

Human Cdc6 is a single copy gene, which encodes a short half-life protein. Abrogation of Cdc6 mRNA may effectively block the cell cycle progression in the proliferative cells. Since the ability to use antisense oligonucleotide (ASO) to selectively target the genetics process involved in cancers had made promising progress, this class of compounds could be developed as novel chemotherapeutic agents. Recent development of anti-bcl-2 ASO as an anti-neoplastic agent is one of many examples of this progress. The efficacy of anti-Cdc6 ASO-(2,5A) to down-regulate Cdc6 mRNA in the human prostate cell line DU145 have been tested. This ASO that contains 18 nucleotides is complimentary to the initiation codon region of Cdc6. It also links a modified oligo 2′, 5′-oligoadenylate (2,5A) to its end (FIG. 3A). The (2,5A), [ppp5′ (AA2′p5′)2A], is a short 2′-5′ linked oligoadenylate which binds to and activates endogenous RNase L. RNase L is an abundant, but dormant enzyme in the cell. Once the RNase L is activated by (2,5A) in the cell, it can cleave single-stranded RNA with moderate specificity for sites 3′ of UpUp and UpAp sequences. It will then lead to degradation of cellular RNA. This improved method combines both actions of conventional ASO and RNase activity, resulting in degradation of a specifically targeted Cdc6 mRNA. FIG. 3B shows that the inviable DU145 cells increased up to ˜20% with 0.25 μM and 0.5 μM of anti-Cdc6 ASO-(2,5A) treatment (lanes 5 & 6). However, little effect was observed from the controls (lane 1, no oligo, lane 2, random, unrelated oligo, lane 3, sense oligo control). The result strongly supported the idea that Cdc6 may be an effective target site for prostate cancer cells.

b) The Use of TUNEL Assay to Examine LNCaP Cells after anti-Cdc6 C-probe Treatment:

If Cdc6 is a good target, any perturbation of Cdc6 mRNA should have a similar inhibition effect. TUNEL assay is used to further investigate the effect of anti-Cdc6 action in prostate cancer cells (TUNEL assay is a popular assay to detect apoptosis or cell programmed death by DNA fragmentation). Initially, prostate cancer cells LNCaP, DU145 and PC-3 are selected for the TUNEL studies to monitor the effect of anti-Cdc6 action. TUNEL assay is a convenient way to detect death cells. Another innovative anti-Cdc6 mRNA method, Covalent anti-Cdc6 cDNA (C-probe), was used for this study. In this method, the antisense single-stranded DNA (ssDNA) was first made. The key step of C-probe preparation was the alkaline hydrolysis of antisense ssDNA in the presence of KMnO₄. The pyrimidine rings (C and T) of the antisense probe was opened and modified to form two additional carboxyl groups. When the anti-Cdc6 C-probe is delivered into cells, it paired to Cdc6 mRNA and formed stable artificial peptide bonds, in place of the normal H-bonds found between G:C and A:T. 0.35 kb N-terminal region of Cdc6 Cprobe (0.35 kb in length) is used. Liposome blank was used as the control. After 24 hour treatment, the anti-Cdc6 C-probe already showed the effect of growth inhibition in a dose dependent manner, whereas control samples were normal. After 96 hour treatment, samples were subjected to TUNEL assay. The untreated blank and liposome treated LNCaP cells showed a normal phenotype. The sense C-probe showed little apoptotic signal, whereas the anti-Cdc6 C-probe treated cells clearly displayed apoptotic phenotype (FIG. 4A). The arrows indicate some positive signals (dark brown spots) due to DNA fragmentation. The result showed that anti-Cdc6 probe was able to effectively induce DNA fragmentation in LNCaP cells.

Similar results were observed in DU145 and PC-3 cells. To further support this finding that the induced DNA fragmentation is due to down-regulation of Cdc6, the Cdc6 protein levels in the presence and absence of anti-Cdc6 probe in all three treated prostate cancer cell lines (FIG. 4B) are examined. The results are consistent with the idea that Cdc6 is a sensitive target in prostate cancer cells. This was the first demonstration that Cdc6 might be the target of induce DNA fragmentation in prostate cancer cells. Since the Cdc6 protein is also decreased accordingly, the simplest explanation for this is that the anti-Cdc6 treatment blocks Cdc6 mRNA, and thus eliminates endogenous Cdc6 protein in the proliferative prostate cancer cells. It is tempting to speculate that this impact may perturb ORC-origin function, halt the initiation of DNA replication, and elicit the checkpoint system to monitor the defect(s). Due to the fact that Cdc6 is a very sensitive target, the repair system may not be able to reverse the defect(s). Thus, the apoptotic machinery was turned on, as the evidence that DNA fragmentation was observed in the present studies. In sum, anti-Cdc6 treatment should be a novel approach to eliminate is highly proliferative cancer cells.

What is claimed is:

1. A method of using Ccd6 as therapeutic, prognostic and diagnostic targets for cell growth/proliferation, comprising the steps of:
(a) collecting test cells, having a Cdc6 mRNA, from a patient as a test cell sample;
(b) providing a control sample having a predetermined control Cdc6 level;
(c) detecting said Cdc6 mRNA from said test cell sample to determine a test Cdc6 level of said test cell sample;
(d) comparing said test Cdc6 level with said control Cdc6 level, to determine an excess Cdc6 mRNA of said patient; and
(e) abrogating said excess Cdc6 mRNA from said patient when said Cdc6 level of said patient is unmatched with said predetermined Cdc6 level.

2. A method of using Ccd6 as therapeutic, prognostic and diagnostic targets for cell growth/proliferation, as recited in claim 1, wherein the step (b) further comprises the steps of:
(b1) collecting control cells from said patient as said control sample; and
(b2) detecting said control Cdc6 mRNA from said control sample to determine said control Cdc6 level.

3. A method of using Ccd6 as therapeutic, prognostic and diagnostic targets for cell growth/proliferation, as recited in claim 1, wherein said Cdc6 mRNA is determined by performing a Northern blot.

4. A method of using Ccd6 as therapeutic, prognostic and diagnostic targets for cell growth/proliferation, as recited in claim 2, wherein said control Cdc6 mRNA is determined by performing a Northern blot.

5. A method of using Ccd6 as therapeutic, prognostic and diagnostic targets for cell growth/proliferation, as recited in claim 1, wherein said excess Cdc6 mRNA is abrogated by an antisense oligonucleotide (ASO).

6. A method of using Ccd6 as therapeutic, prognostic and diagnostic targets for cell growth/proliferation, as recited in claim 2, wherein said excess Cdc6 mRNA is abrogated by an ASO.

7. A method of using Ccd6 as therapeutic, prognostic and diagnostic targets for cell growth/proliferation, as recited in claim 3, wherein said excess Cdc6 mRNA is abrogated by an ASO.

8. A method of using Ccd6 as therapeutic, prognostic and diagnostic targets for cell growth/proliferation, as recited in claim 4, wherein said excess Cdc6 mRNA is abrogated by an ASO.

9. A method of using Ccd6 as therapeutic, prognostic and diagnostic targets for cell growth/proliferation, as recited in claim 5, wherein said ASO is anti-bcl-2 antisense oligonucleotide.

10. A method of using Ccd6 as therapeutic, prognostic and diagnostic targets for cell growth/proliferation, as recited in claim 6, wherein said ASO is anti-bcl-2 antisense oligonucleotide.

11. A method of using Ccd6 as therapeutic, prognostic and diagnostic targets for cell growth/proliferation, as recited in claim 7, wherein said ASO is anti-bcl-2 antisense oligonucleotide.

12. A method of using Ccd6 as therapeutic, prognostic and diagnostic targets for cell growth/proliferation, as recited in claim 8, wherein said ASO is anti-bcl-2 antisense oligonucleotide.

13. A method of using Ccd6 as therapeutic, prognostic and diagnostic targets for cell growth/proliferation, as recited in claim 5, wherein said ASO is anti-Cdc6 antisense oligonucleotide-(2,5)A.

14. A method of using Ccd6 as therapeutic, prognostic and diagnostic targets for cell growth/proliferation, as recited in claim 6, wherein said ASO is anti-Cdc6 antisense oligonucleotide-(2,5)A.

15. A method of using Ccd6 as therapeutic, prognostic and diagnostic targets for cell growth/proliferation, as recited in claim 7, wherein said ASO is anti-Cdc6 antisense oligonucleotide-(2,5)A.

16. A method of using Ccd6 as therapeutic, prognostic and diagnostic targets for cell growth/proliferation, as recited in claim 8, wherein said ASO is anti-Cdc6 antisense oligonucleotide-(2,5)A.
